(19) United States
(12) Patent Application Publication
(10) Pub. No.: US 2012/0052043 A1
(43) Pub. Date: Mar. 1, 2012

(54) Chemokine-Chaperone Fusion Proteins

(76) Inventor: Andreas Kungl, Graz (AT)

(21) Appl. No.: 12/519,430

(22) PCT Filed: Dec. 20, 2007
(86) PCT No.: PCT/AT2007/000577

§ 371 Date: Jun. 16, 2009

(30) Foreign Application Priority Data
Dec. 20, 2006 (AT) A2101/2006

(51) Int. Cl. A61K 38/19; A61K 31/7088; C12N 15/63; C12N 5/00; C07K 14/52; C07H 21/04

(52) U.S. Cl. 424/85.1; 530/351; 536/23.4; 435/320.1; 435/325; 514/44 R

(57) ABSTRACT

A fusion protein comprising at least one chemokine or a derivative or fragment thereof and at least one peptide derived from a chaperone. The fusion protein can be provided in a pharmaceutical preparation for the treatment of inflammatory conditions or cancer.

### CROSS-REFERENCE TO RELATED APPLICATIONS

This application is the U.S. national stage of International Patent Application No. PCT/AT2007/000577, filed Dec. 20, 2007 and entitled CHEMOKINE-CHAPERONE FUSION PROTEINS, which claims the benefit of priority from Austrian Patent Application No. A 2101/2006, filed Dec. 20, 2006. The disclosures of the foregoing applications are incorporated herein by reference in their entirety.

The invention relates to novel fusion proteins for engineering T-cell responsiveness into chemokines establishing additional protection in inflammatory conditions. Further, the invention relates to a method for production of said fusion protein and its use for therapeutic purposes especially in the field of inflammation or cancer treatment.

Vertebrates have the ability to establish an immune response as a defence against pathogens from the environment as well as against aberrant cells, such as tumour cells, which develop internally. This can take the form of innate immunity, which is mediated by NK cells, neutrophils and cells of the monocyte/macrophage lineage, or the form of acquired or active immunity against specific antigens mediated by lymphocytes. Active immune responses can be a humoral response which entails the production of specific antibodies that serve to neutralise antigens exposed to the systemic circulation and aid in their uptake by phagocytic cells, and a cellular response which is required for recognition of infected or aberrant cells within the body. Often, this immunogenic response results in diseases and disorders that cause harm to the organism itself. Such disorders are associated with the recognition of self proteins and cells as foreign and, thus, trigger an attack of such cells or cellular proteins. Common autoimmune disorders include, for example, psoriasis, rheumatoid arthritis, lupus, diabetes and other diseases known in the art.

The pathogenesis of an autoimmune disease may begin with abnormal regulation of autoreactive T cells either due to bystander activation or due to molecular mimicry. For example, a viral infection or exposure to a superantigen may provide sufficient co-stimulation resulting in activation of few low affinity autoreactive T cells that escape the thymus selection. Abnormal down-regulation of such autoreactive responses may lead to expansion of pathogenic T cells that infiltrate the organ where the recognised antigen is present. Local inflammation and direct destruction of host cells trigger antigen release, uptake by APCs and presentation to specific T cells, thus providing a positive feed-back that exacerbates the autoimmunity. Simultaneously, normally cryptic, organ-associated antigens may become exposed in the context of activation of professional antigen presenting cells and antigen release, resulting in activation of T cells specific for these other self antigens. Particularly in conditions favouring overall Th1/Th2 imbalance, the employment of additional specificities may accelerate the disease. It is widely believed that whereas Th1 cytokines such as IFN-kappa contribute to the pathogenesis of autoimmunity, Th2 cytokines such as IL-4 and IL-10 may suppress the activity of pathogenic Th1 cells.

Heat shock proteins (Hsp's) belong to the large chaperone protein family. They are ubiquitous, highly conserved proteins with significant interspecies homologies that play an important role in various cellular processes. They are immunodominant stress proteins that are upregulated during cellular stress. Those unique qualities of Hsp's (evolutionary conservation, immunodominance and upregulation during stress) have made Hsp's attractive candidates as targets for immunotherapy and vaccines (Puga Yung G. L., 2003, Inflamm. res., 443-451). Indeed, at present, the role of immune reactivity to Hsp's has been proposed in different disease models, such as infectious diseases and autoimmune diseases. Most evidence for the role of Hsp's in the immune regulation of inflammatory diseases comes from models of chronic arthritis. It has been shown that immunisation with hsp10, hsp60 and hsp70 can confer protection in virtually all models of experimental arthritis. In the model of adjuvant arthritis, immune reactivity to Hsp's plays a role both in the induction of disease and in protection from disease. On the one hand, it was shown that adjuvant arthritis can be induced by means of a T cell clone that is specific for mycobacterial Hsp60 180-188. On the other hand, later studies showed that preimmunisation with mycobacterial Hsp60 can effectively protect against disease induction. However, after immunisation with Hsp60, several epitopes were found to be recognised by the immune system. Interestingly, only one epitope (Hsp 60 aa256-270) out of eight epitopes was found to be capable of inducing protection (Anderton S. et al., J. Exp. Med., 1995, pp. 943-952). This protection was based on the induction of cross-reactive T cells.

Over the last years it has become clear that immune reactivity to Hsp's also plays a crucial role in human chronic arthritis, namely Juvenile Idiopathic Arthritis (JIA) and Rheumatoid Arthritis (RA). First, an increased expression of Hsp60 was detected in synovial lining cells of subjects with JIA and RA. Secondly, T cell reactivity to both self and non-self hsp60 was found in both diseases. Similarly, immune reactivity to other hsps such as Hsp70 and Dnaj was detected in subjects with JIA and RA.

Treatment strategies for many diseases are directed at alleviating the symptoms of the disease rather than resolving the cause of the problematic symptoms. In the case of inflammatory diseases, for example, most treatments are directed at relieving the inflammation generally such as by using steroidal or non-steroidal anti-inflammatory agents.

Inflammation often occurs as a result of an immune response. Although an immune response and the consequent inflammatory response generally provide an advantage to an individual, for example, where the response is to a bacterial infection, in some cases an immune response and inflammatory response produce deleterious consequences. In particular, patients with an auto-immune disease, such as rheumatoid arthritis, systemic lupus erythematosis, and the like, often suffer from severe and in some cases generalised tissue damage. Although administration of a steroidal drug, for example, can decrease the severity of the immune response in these patients, the long-term use of such drugs can lead to adverse effects. Furthermore, the use of such drugs generally reduces the ability of an individual to mount an immune response, leaving the individual susceptible to short-term infections that can produce severe consequences.

So although the use of immunogenic peptides, esp. of peptides derived from heat shock proteins, act via a T cell mediated immune response, result in increased expression of pro-inflammatory cytokines such as interferon-gamma, resulting in increased expression of anti-inflammatory cytokines such as interleukin-10. A drawback of using these peptides is the fact that the immune response, and thereby the intended therapeutic effect, is delayed due to the need to switch on the T cell response mechanism.

Thus, a need exists for compositions and methods that are useful for specifically modulating an immune response that leads to long-term protection and, additionally, to provide means for immediately inducing a therapeutic effect in inflammatory diseases.

The aim of the present invention is therefore to provide a system, wherein the broad and long-lasting therapeutic effect of Hsp-derived peptides for the treatment of inflammatory diseases is supplemented by an immediate therapy.

According to the present invention a T-cell responsiveness can be engineered into dominant-negative mutant chemokines—i.e. knocked-in glycosaminoglycan binding and knocked-out GPCR activity—establishing additional, such as adaptive immunity-based, protection in inflammatory conditions.

The present invention provides a fusion protein comprising at least one chemokine or a derivative or fragment thereof and at least one peptide derived from a chaperone. Preferably, the chaperones are T-cell epitopes of heat shock proteins, preferably derived from Hsp60, Hsp65 and Dnaj1. In a preferred embodiment of the invention, the chemokine is a modified chemokine having increased GAG binding affinity and, optionally, a knocked-out or down-regulated further biological activity compared to the wild-type chemokine protein. The present invention further comprises an isolated polynucleic acid molecule coding for said fusion protein and a fusion protein expression vector.

For therapeutic purposes, a pharmaceutical composition comprising a fusion protein according to the invention, or a polynucleic acid or a vector and a pharmaceutically acceptable carrier is also provided by the present invention.

The chemokine or derivative or fragment thereof which is part of the fusion protein according to the invention can be any chemokine as known in the art.

Preferably, the chemokine is IL-8, RANTES, SDF-1, I-TAC or MCP-1 or a derivative or fragment thereof. Yet, a chemokine molecule is preferred that shows increased binding affinity to GAG and has optionally also a knocked-out or down-regulated G-protein coupled receptor (GPCR) activity. Chemokines that have been modified accordingly have been described in WO 05/054285 and in Potzinger et al. (Biochem. Soc. Trans. 34, 435-437 (2006)). These chemokines have been modified by substitution, insertion, and/or deletion of at least one amino acid in order to increase the relative amount of basic or electron-donating amino acids in the GAG binding region, and/or to reduce the amount of bulky and/or acidic amino acids in the GAG binding region preferably at a solvent exposed position. Preferably, at least one basic or electron-donating amino acid selected from the group consisting of Arg, Lys, and His, Asn, Gin is inserted into said GAG binding region of said chemokine. Preferably, GAG binding affinity is an increased binding affinity to chondroitin sulfate, heparan sulfate, keratan sulfate and/or heparin.

Derivatives or fragments according to the invention can be chemokines being truncated or modified but still showing at least 5-time increased GAG binding affinity, preferred 10-time increased GAG binding affinity, still preferred 100-time increased GAG binding affinity of the chemokine activity of the unmodified protein.

In a specific embodiment the chemokine is interleukin 8 (IL-8), wherein positions 17, 21, 70, and/or 71 in IL-8 are substituted by Arg, Lys, His, Asn and/or Gin, preferably all four positions (17, 21, 70 and 71) are substituted by Lys.

The chemokine molecules as used in the fusion protein according to the invention, can comprise a further biologically active region, which is modified, thereby knocking-out or down-regulating the GPCR activity of said protein by deletion, insertion, and/or substitution, for example with alanine, a sterically and/or electrostatically similar residue.

In case of IL-8, said further biologically active region is located within the first 10 N-terminal amino acids, and, therefore, preferably the first 6 N-terminal amino acids are deleted.

Most preferred is an IL-8 mutant that comprises the structure IL-8 (Δ6 F17K F21K E70K N71K), IL-8 (Δ6 E70K N71K) or IL-8 (Δ6 E70R), IL-8 (Δ6F17R E70R N71K), IL-8 (del6F17RE70KN71R) and IL-8 (A 6E70K N71K).

Alternatively, the chemokine can be MCP1 or a derivative or fragment thereof. According to a specific embodiment of the invention the fusion protein can comprise a MCP1 that can be a mutant with increased glycosaminoglycan (GAG) binding affinity and knocked-out or reduced GPCR activity compared to wild type MCP-1. For example, it can be a mutant of the following structure:

(M)ₙQ(PDAINAP)ₘVTCC(X1)NFTN RKI(X2)V(X3)RLAS
YRRITSSKCP KEAVIFKTI(X4) AKEICADPKQ KWVQDSMDHL
DKQTQTPKT

wherein X1 is selected of the group consisting of Y and/or A, preferably it is A,
wherein X2 is selected of the group consisting of S, R, K, H, N and/or Q, preferably it is K,
wherein X3 is selected of the group consisting of Q, R, K, H, N and/or Q, preferably it is R,
wherein X4 is selected of the group consisting of V, R, K, H, N and/or Q, preferably it is K,
and wherein n and/or m can be either 0 or 1.

Even more specifically, it can be selected from the group of Met-MCP-1 Y13A S21K, Met-MCP-1 Y13A S21K V47K, Met-MCP-1 Y13A S21K Q23R and Met-MCP-1 Y13A S21K Q23R V47K.

As a further alternative part of the fusion protein can be SDF-1 or a fragment or derivative thereof. For example, it can be SDF-1α or SDF-1β or SDF-1 gamma or any variant thereof which exhibit (i) increased glycosaminoglycan (GAG) binding affinity and (ii) inhibited or down-regulated GPCR activity compared to wild type SDF-1.

According to a specific embodiment it can be described by the general formula:

(M)ₙ(X1)ₘ(X2)ₚVSLSYRCPCRFFESHVARANVKHLKI(X3)NTPNCA
LQI(X4)ARLKNNNRQVCIDPKLKWIQEYLEKALNK(GRREEKVGKKEKI
GKKKRQKKRKAAQKRKN)ₒ

wherein X1 is a Lysine or Arginine residue,
wherein X2 is a Proline or Glycine residue,
wherein X3 is selected of the group consisting of Y and/or A, preferably it is A,
wherein X4 is selected of the group consisting of S, R, K, H, N and/or Q, preferably it is K,
and wherein n and/or m and/or p and/or o can be either 0 or 1.

According to an alternative embodiment of the invention the SDF-1 mutant protein can contain an N-terminal Met.

In a further alternative embodiment, the chemokines can also be derivatives or fragments of RANTES or MCP1 as for example described in WO 03/84993, WO 02/28419 or EP0828833.

According to the invention the fusion protein comprises at least one peptide derived from a chaperone. Chaperones are large multisubunit proteins whose primary function is to assist other proteins in achieving proper folding and/or to avoid improper aggregation. Among the chaperone family are heat shock proteins major members, i.e. proteins expressed in response to elevated temperatures and/or other cellular stress. This is severely affected by heat and, therefore, some chaperones act to repair the potential damage caused by misfolding. Other chaperones are involved in folding newly made proteins as they are extruded from the ribosome.

Molecular chaperones are well known in the art, several families thereof being characterised. These chaperones can be, for example, p90 Calnexin, Hsp family, DNA K, DNA J, Hsp60 family, GroEL, ER-associated chaperones, Hsp65, Hsp90, Hsc70, sHsps, SecA, SecB, Trigger factor, zebrafish hsp47, 70 and 90, Hsp47, GRP94, Cpn10, BiP, GRP78, Cl p, FtsH, Ig invariant chain, mitochondrial hsp70, EBP, mitochondrial m-AAA, yeast Ydj1, Hsp104, ApoE, Syc, Hip, TriC family, CCT, PapD, calmodulin etc.

Preferably, the chaperones according to the present invention are T-cell peptides derived from heat shock proteins, preferably from bacterial or other microbial origin.

According to the present invention, T-cell epitopes from all heat shock proteins can be used that are known in the art. Among these are Hsp10, Hsp60, Hsp65, Dnaj, Hsp70; Dnaj and Hsp65 being preferred. In a further preferred embodiment, the T-cell epitope is identical to the sequence ALSTLVVNKI (SEQ ID No.1) or has at least 90% identity.

The peptide derived from a chaperone can be of a length of up to 50 amino acids, preferably up to 30 amino acids, preferably up to 12 amino acids, preferably up to 10 amino acids, preferably up to 8 amino acids. Peptides are selected based on their HLA (Human Leukocyte Antigen) avidity and, therefore, on their ability to induce a T-cell response.

The fusion protein according to the present invention preferably comprises one peptide derived from a chaperone, alternatively, it may contain two chaperone-derived peptides, one at the N- and one at the C-terminus of the fusion protein construct.

According to the invention the chemokine and at least one peptide derived from a chaperone are either fused directly together or with a peptide linker sequence. The peptide linker sequence is preferably of 27 amino acids length, still preferably of 8 amino acids length, still preferably of 4 amino acids length.

A further aspect of the present invention is an isolated polynucleic acid molecule which codes for the inventive protein as described above. The polynucleic acid may be DNA or RNA. Thereby the modifications which lead to the inventive fusion protein are carried out on DNA or RNA level. This inventive isolated polynucleic acid molecule is suitable for diagnostic methods as well as gene therapy and the production of inventive fusion protein on a large scale.

Still preferred, the isolated polynucleic acid molecule hybridises to the above defined inventive polynucleic acid molecule under stringent conditions. Depending on the hybridisation conditions complementary duplexes form between the two DNA or RNA molecules, either by perfectly matching or also comprising mismatched bases (see Sambrook et al., Molecular Cloning: A laboratory manual, 2^{nd} ed., Cold Spring Harbor, N.Y. 1989). Probes greater in length than about 50 nucleotides may accomplish up to 25 to 30% mismatched bases. Smaller probes will accomplish fewer mismatches. The tendency of a target and probe to form duplexes containing mismatched base pairs is controlled by the stringency of the hybridisation conditions which itself is a function of factors, such as the concentrations of salt or formamide in the hybridisation buffer, the temperature of the hybridisation and the post-hybridisation wash conditions. By applying well known principles that occur in the formation of hybrid duplexes conditions having the desired stringency can be achieved by one skilled in the art by selecting from among a variety of hybridisation buffers, temperatures and wash conditions. Thus, conditions can be selected that permit the detection of either perfectly matching or partially matching hybrid duplexes. The melting temperature (Tm) of a duplex is useful for selecting appropriate hybridisation conditions. Stringent hybridisation conditions for polynucleotide molecules over 200 nucleotides in length typically involve hybridising at a temperature 15-25° C. below the melting temperature of the expected duplex. For olignucleotide probes over 30 nucleotides which form less stable duplexes than longer probes, stringent hybridisation usually is achieved by hybridising at 5 to 10° C. below the Tm. The Tm of a nucleic acid duplex can be calculated using a formula based on the percent G+C contained in the nucleic acids and that takes chain lengths into account, such as the formula Tm=81.5-16.6(log [NA+])+0.41 (% G+C)−(600/N), where N=chain length.

A further aspect of the present invention relates to a vector which comprises an isolated polynucleic molecule, preferably a DNA molecule according to the present invention as defined above. The vector comprises all regulatory elements necessary for efficient transfection as well as efficient expression of proteins. Such vectors are well known in the art and any suitable vector can be selected for this purpose. Typically, pET-derived vectors are used for subcloning of the constructs such as the TOPO plasmids provided by Invitrogen.

A further aspect of the present application relates to a recombinant cell or cell line which is transfected with an inventive vector as described above. Such recombinant cells as well as any descendant cell comprise the vector. Thereby a cell line is provided which expresses the modified protein either continuously or upon activation depending on the vector.

The cells used according to the invention are preferably animal cells, more preferably mammalian cells. These can be, for example, BSC-1 cells, LLC-MK cells, CV-1 cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO, perC6 (human retina cells) or derivatives thereof.

Alternatively, also bacterial cells like BL21 (DE3) can be used for expression.

A further aspect of the present invention relates to a pharmaceutical composition which comprises a protein, a polynucleic acid or a vector according to the present invention as defined above and a pharmaceutically acceptable carrier. Of course, the pharmaceutical composition may further comprise additional substances which are usually present in pharmaceutical compositions, such as salts, buffers, emulgators, colouring agents, etc.

A further aspect of the present invention relates to the use of the modified protein, a polynucleic acid or a vector according to the present invention as defined above in a method for treatment of inflammatory diseases.

According to the invention, the treatment of all inflammatory diseases are covered herein, in which an infiltration of leukocytes into a diseased site occurs, or diseases in which an inflammation markedly occurs due to active oxygen and various cytokines which are released from leukocytes. Examples of inflammatory diseases are rheumatoid arthritis, psoriasis, osteoarthritis, asthma, COPD, multiple sclerosis, ulcerative colitis and Crohn's disease.

Additionally, inflammatory diseases like uveitis, inflammatory bowel disease, myocardial infarction, congested heart failure or ischemia reperfusion injury can be treated using a fusion protein according to the invention, for example using an MCP1 mutant with increased GAG binding affinity.

As outlined above, the modified protein can act as chemokine-based glycosaminoglycan antagonist (i.e. displace wild-type chemokines from GAG co-receptors) with immediate action in inflammatory reactions and by inducing long-lasting T cell response (with delayed onset) due to the presence of the chaperone moiety.

For treatment of cancer, a fusion protein is preferred wherein SDF-1 or part thereof is fused to a chaperone molecule.

The following examples and figures describe the invention in more detail without limiting the scope of the invention

### FIGURES

FIG. 1: Amino acid sequence of the Hsp60 peptide/PA401 fusion construct, SEQ ID NO 2.

FIG. 2: Expression vector map. Gene of the fusion protein was subcloned into the expression plasmid using the Topo-Isomerase system provided by Invitrogen.

FIG. 3: Expression analysis of fusion protein PA515.

FIG. 4: Inhibitory activity of the fusion protein PA515 in a murine neutrophil leukocyte infiltration model.

### EXAMPLES

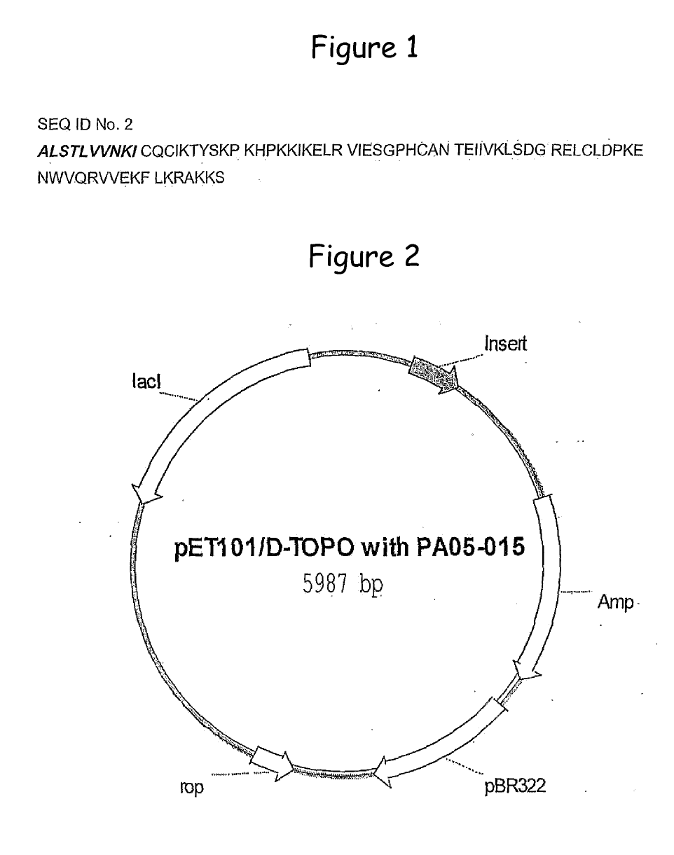

The fusion protein consisting of an hsp65-derived peptide and the IL-8 mutant PA401 (PA 04-001, PA 04) was cloned (the amino acid sequence of the fusion protein is displayed in FIG. 1). For generating the N-terminal fusion protein termed PA515, the gene for the construct was obtained by PCR-amplifying the cDNA of PA401 from its expression plasmid using a forward primer which contains:
the bp sequence for the ALSTLVVNKI hsp65-derived peptide
followed by the 5′ part of the PA401 gene.

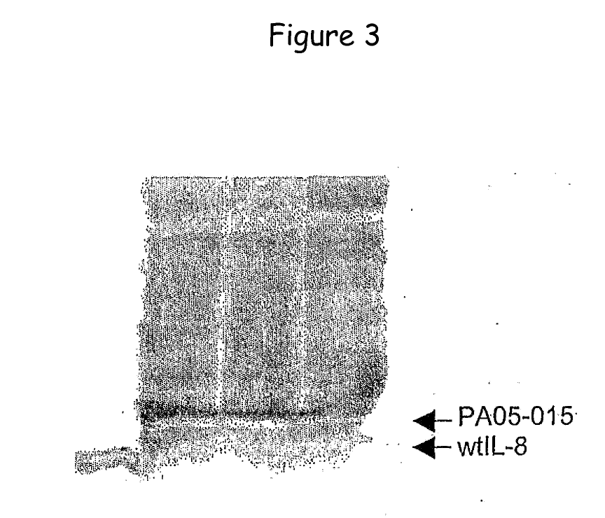

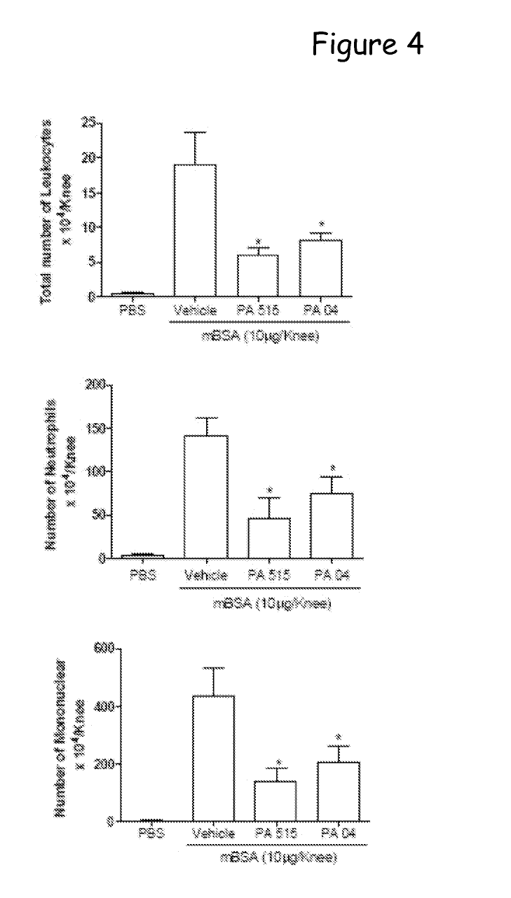

The 3′ part of the PA401 gene was unmodified. The resulting PCR product was subsequently sub-cloned into the D-TOPO expression plasmid (see FIG. 2). After positive sequencing of the construct, it was transformed into BL21 (DE3) cells for fusion protein expression. Protein expression was found to be sufficiently high for detection by Coomassie Blue staining (FIG. 3).

The biological activity of the new fusion protein PA515 was tested in a murine model for acute rheumatoid arthritis. For this purpose, animals (five per group) were immunised with methyl BSA (MBSA) in Freud's complete adjuvant. 14 days after immunisation, the animals were challenged with MBSA in the knee joint. The compounds (the mutant PA515 and the control IL-8 mutant PA04) were given at 0.1 μg/mouse 30 min before, followed by 1 hr and 6 hrs after the challenge. Cell influx was evaluated by FACS analysis after 24 hrs. The inhibitory effect of the mutant PA515 was slightly better in this model compared to the parent mutant PA401. However, based on the structure of the PA515 mutant, we expect a different kinetics of the biological effect of PA515 compared to PA401. This means that due to the fusion with the hsp65-derived peptide, a much longer lasting inhibition of neutrophil/leukocyte migration is predicted.

1. Fusion protein comprising (i) at least one chemokine or a derivative or fragment thereof and (ii) at least one peptide derived from a chaperone.

2. Fusion protein according to claim 1, characterised in that the chaperone is a heat shock protein.

3. Fusion protein according to claim 1, characterised in that the chemokine is IL-8, RANTES, SDF-1, I-TAC or MCP1 or a derivative or fragment thereof.

4. Fusion protein according to claim 1, characterised in that the chemokine is modified towards increased GAG binding affinity compared to the GAG binding affinity of a respective wild-type protein.

5. Fusion protein according to claim 4, characterised in that the intrinsic GAG binding region is modified by substitution, insertion, and/or deletion of at least one amino acid in order to increase the relative amount of basic or electron-donating amino acids in said GAG binding region, and/or reduce the amount of bulky and/or acidic amino acids in said GAG binding region, preferably at a solvent exposed position.

6. Fusion protein according to claim 5, characterised in that at least one basic amino acid selected from the group consisting of Arg, Lys, His, Asn and Gln is inserted into said GAG binding region of said chemokine.

7. Fusion protein according to claim 4, characterised in that said GAG binding region is a C terminal-helix.

8. Fusion protein according to claim 1, characterised in that GPCR activation of the fusion protein is inhibited or down-regulated by deletion, insertion, and/or substitution of at least one amino acid in said region.

9. Fusion protein according to claim 1, characterised in that the chemokine is IL-8 and that positions 17, 21, 70, and/or 71 are substituted by Arg, Lys, His, Asn and/or Gln, preferably all four positions are substituted by Lys.

10. Fusion protein according to claim 1, characterised in that the chemokine is an IL-8 mutant with the first 6 N-terminal amino acids deleted.

11. Fusion protein according to claim 1, characterised in that the chemokine is an IL-8 mutant selected from the group consisting of IL-8 (Δ6 F17K F21K E70K N71K), IL-8 (Δ6 E70K N71K) or IL-8 (Δ6 E70R), IL-8 (Δ6F17R E70R N71K), IL-8 (Δ6F17RE70KN71R) and IL-8 (Δ6E70K N71K).

12. Fusion protein according to claim 1 characterised in that the chemokine is MCP1 of the following structure:
(M)ₙQ(PDAINAP)ₘVTCC(X1)NFTN RKI(X2)V(X3)RLAS
YRRITSSKCP KEAVIFKTI(X4) AKEICADPKQ KWVQDSMDHL
DKQTQTPKT,
wherein X1 is selected of the group consisting of Y and/or A, preferably it is A,
wherein X2 is selected of the group consisting of S, R, K, H, N and/or Q, preferably it is K,
wherein X3 is selected of the group consisting of Q, R, K, H, N and/or Q, preferably it is R,
wherein X4 is selected of the group consisting of V, R, K, H, N and/or Q, preferably it is K,
and wherein n and/or m can be either 0 or 1.

13. Fusion protein according to claim 1 characterised in that the chemokine is an MCP1 mutant selected from the group consisting of Met-MCP-1 Y13A S21K, Met-MCP-1 Y13A S21K V47K, Met-MCP-1 Y13A S21K Q23R and Met-MCP-1 Y13A S21K Q23R V47K.

14. Fusion protein according to claim 1 characterised in that the chemokine is SDF-1 of the following structure:
(M)ₙ(X1)ₘ(X2)ₚVSLSYRCPCRFFESHVARANVKHLKI(X3)NTPNCA
LQI(X4)ARLKNNNRQVCIDPKLKWIQEYLEKALNK(GRREEKVGKKEKI
GKKKRQKKRKAAQKRKN)ₒ
wherein X1 is a Lysine or Arginine residue,
wherein X2 is a Proline or Glycine residue,
wherein X3 is selected of the group consisting of Y and/or A, preferably it is A,
wherein X4 is selected of the group consisting of S, R, K, H, N and/or Q, preferably it is K,
and wherein n and/or m and/or p and/or o can be either 0 or 1.

15. Fusion protein according to claim 1, characterised in that the chaperone is a T-cell epitope of a heat shock protein.

16. Fusion protein according to claim 15, characterised in that the T-cell epitope is derived from hsp60, hsp65 or dnaj1 protein.

17. Fusion protein according to claim 1, characterised in that the peptide is at least 8 amino acids in length, preferably at least 10 amino acids, preferably at least 12 amino acids.

18. Fusion protein according to claim 1, characterised in that the peptide comprises an amino acid sequence of SEQ ID No 1.

19. Fusion protein according to claim 1, characterised in that the chemokine and the chaperone are fused via a peptide linker sequence.

20. Isolated polynucleic acid molecule, characterised in that it codes for a fusion protein according to claim 1.

21. Isolated polynucleic acid molecule, characterised in that it hybridises to the DNA molecule according to claim 20 under stringent conditions.

22. Vector, characterised in that it comprises an isolated polynucleic molecule according to claim 20 or an isolated polynucleotide molecule according to claim 21.

23. Vector, characterised in that it comprises a nucleic acid sequence of SEQ ID No.2.

24. Recombinant cell, characterised in that it is transfected with a vector according to claim 22 or a vector according to claim 23.

25. Pharmaceutical composition, characterised in that it comprises a fusion protein according to claim 1, a polynucleic acid according to claim 20 or a vector according to claim 22 and a pharmaceutically acceptable carrier.

26. A method of treating an inflammatory condition or cancer, comprising administering the fusion protein according to claim 1, a polynucleic acid according to claim 20 or a vector according to claim 22.

27. The method according to claim 26, characterised in that the inflammatory condition is selected from a the group consisting of rheumatoid arthritis, psoriasis, osteoarthritis, asthma, COPD, multiple sclerosis, ulcerative colitis and Crohn's disease, uveitis, inflammatory bowel disease, myocardial infarction, congested heart failure and ischemia reperfusion injury.


SEQUENCE LISTING (4 sequences)

SEQ ID NO: 1 - protein, 10 aa, Artificial Sequence
  T cell epitope
ALSTLVVNKI

SEQ ID NO: 2 - protein, 76 aa, Artificial Sequence
  Hsp60 peptide/PA401 fusion construct
ALSTLVVNKICQCIKTYSKPKHPKKIKELRVIESGPHCANTEIIVKLSDGRELCLDPKENWVQRVVEKFLKRAKKS

SEQ ID NO: 3 - protein, 77 aa, Artificial Sequence
  mutated MCP-1 protein
MQPDAINAPVTCCXNFTNRKIXVXRLASYRRITSSKCPKEAVIFKTIXAKEICADPKQKWVQDSMDHLDKQTQTPKT

SEQ ID NO: 4 - protein, 99 aa, Artificial Sequence
  SDF-1 mutated protein
MXXVSLSYRCPCRFFESHVARANVKHLKIXNTPNCALQIXARLKNNNRQVCIDPKLKWIQEYLEKALNKGRREEKVGKKEKIGKKKRQKKRKAAQKRKN